(19) United States
(12) Patent Application Publication
(10) Pub. No.: US 2026/0194445 A1
(43) Pub. Date: Jul. 9, 2026

(54) DETERMINING CORROSION RISK USING RISK-BASED MATRICES

(71) Applicant: SAUDI ARABIAN OIL COMPANY, Dhahran (SA)

(72) Inventors: Abdullah ALRATOEE, Dhahran (SA); Yousef ALDOSSARY, Dhahran (SA); Omar ALABDULGADER, Dhahran (SA)

(73) Assignee: SAUDI ARABIAN OIL COMPANY, Dhahran (SA)

(21) Appl. No.: 19/013,439

(22) Filed: Jan. 8, 2025

(51) Int. Cl. G01N 17/02; G01N 33/18

(52) U.S. Cl. CPC G01N 17/02; G01N 33/18

(57) ABSTRACT

Methods for assessing corrosion risk may monitor a plurality of parameters in a water stream. The plurality of parameters comprises pH, temperature, oxygen content, and conductivity. A first corrosion risk factor is assessed using a first risk-based matrix based upon pH as a function of temperature, and a second corrosion risk factor is assessed using a second risk-based matrix based upon oxygen content as a function of conductivity. The first corrosion risk factor is assigned as a pH low-risk, a pH medium-risk, or a pH high-risk, and the second corrosion risk factor is assigned as an oxygen content low-risk, an oxygen content medium-risk, or an oxygen content high-risk. An overall corrosion risk factor is determined as a higher of the first corrosion risk factor and the second corrosion risk factor. If the overall corrosion risk factor is not low-risk, a corrective action is performed to decrease the overall risk factor.

### FIELD OF THE DISCLOSURE

The present disclosure relates generally to corrosion and, more particularly, to methods for assessing corrosion risk.

### BACKGROUND OF THE DISCLOSURE

Sour water is water containing an acid, specifically hydrogen sulfide (H₂S). Sour water is often formed through contact of water with petroleum and/or petroleum derivatives in which the hydrogen sulfide is already present or formed through metabolic processes of anaerobic microorganisms. The acidity of sour water can lead to corrosion of metal surfaces, such as tubulars, downhole tools, storage vessels, pipelines, and the like. Corrosion involves oxidation-reduction reactions of a metal or metal alloy and a corrosive agent, such as hydrogen sulfide in the case of sour water. Examples of corrosion damage may include, but are not limited to, rusting, metal dissolution or erosion, pitting, peeling, blistering, patina formation, combinations thereof, and the like. If not effectively managed, corrosion may lead to serious issues, such as structural failure and/or process downtime.

Corrosion commonly may be monitored using equipment such as corrosion probes and test coupons. Although techniques using such equipment are effective for assessing corrosion, corrosion monitoring may involve invasive sampling and involve time-consuming analysis procedures. Because corrosion analyses are not conducted in near real-time, corrosion may have already become problematic by the time it is determined corrosive conditions are present. In the case of invasive sampling, there is also a risk of leakage during test coupon retrieval.

Multiple physical and chemical properties may impact the severity of corrosion under specified conditions. In some cases, corrosion inhibitors may be introduced to a water stream to discourage corrosion when conditions are otherwise corrosion-favorable. In other cases, a water stream may be treated to alter one or more corrosion-causing conditions to decrease the propensity toward corrosion. Because corrosion is such a serious issue, actions are commonly taken to mitigate all possible corrosion-causing conditions as much as possible, especially in view of the slow rate at which traditional corrosion analyses may be performed. Although the foregoing tactics may be effective for addressing corrosion, this excessively cautious approach may result in unneeded use of chemicals and/or time-consuming mitigation processes when they may not actually be necessary.

### SUMMARY OF THE DISCLOSURE

Various details of the present disclosure are hereinafter summarized to provide a basic understanding. This summary is not an extensive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

According to an embodiment consistent with the present disclosure, a method comprising: monitoring a plurality of parameters in a water stream; wherein the plurality of parameters comprises pH, temperature, oxygen content, and conductivity; assessing a first corrosion risk factor using a first risk-based matrix based upon pH as a function of temperature; assigning the first corrosion risk factor as a pH low-risk, a pH medium-risk, or a pH high-risk; assessing a second corrosion risk factor using a second risk-based matrix based upon oxygen content as a function of conductivity; assigning the second corrosion risk factor as an oxygen content low-risk, an oxygen content medium-risk, or an oxygen content high-risk; assessing an overall corrosion risk factor as a higher of the first corrosion risk factor and the second corrosion risk factor, the overall corrosion risk factor being an overall low-risk, an overall medium-risk, or an overall high-risk; wherein if the first corrosion risk factor and the second corrosion risk factor are both a medium-risk the overall corrosion risk factor is designated as overall medium-risk, and if the first corrosion risk factor and the second corrosion risk factor are both a high-risk, the overall corrosion risk factor is designated as overall high-risk; and if the overall risk factor is assessed as an overall high-risk or an overall medium-risk, performing at least one corrective action upon the water stream to decrease the overall risk factor.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

The following figures are included to illustrate certain aspects of the embodiments. The subject matter disclosed is capable of considerable modifications, alterations, combinations and equivalents in form and function, as will occur to those skilled in the art and having the benefit of this disclosure.

FIG. 1 is an illustrative example of a risk-based matrix based on pH as a function of temperature.

FIG. 2 is an illustrative example of a risk-based matrix based on oxygen content as a function of conductivity.

FIG. 3 is a diagram of an illustrative system for monitoring and processing sour water according to embodiments of the present disclosure.

### DETAILED DESCRIPTION

Embodiments in accordance with the present disclosure generally relate to corrosion control and, more particularly, to methods for assessing corrosion risk and determining corrective action.

As discussed above, traditional approaches for addressing corrosion may utilize invasive sampling techniques that may provide a delayed assessment of ongoing corrosion. This approach may result in unneeded use of chemicals and/or time-consuming mitigation processes when they may not actually be necessary

In response to the foregoing, the present disclosure leverages conventionally measured process parameters to provide an ongoing assessment of the risk of corrosion. Namely, the present disclosure evaluates the risk of corrosion based upon the values of various process parameters and how these parameters individually or collectively contribute to corrosion. Additional details of how the process parameters are evaluated and adjusted to mitigate corrosion are discussed hereinafter.

Oxygen content and pH are two process parameters that may significantly impact corrosion. However, the extent to which oxygen content and pH lead to corrosion differ based upon other process parameters, namely temperature in the case of pH and conductivity in the case of oxygen content.

In the present disclosure, multiple risk-based matrices may be utilized to assess the individual contributions of pH and oxygen content to corrosion risk under specified conditions, as well as the overall risk of corrosion when taking both process parameters into account. In each risk-based matrix, the individual sets of process conditions (i.e., pH as a function of temperature, and oxygen content as a function of conductivity) may be gridded in a matrix over analysis ranges of interest to create a plurality of cells, each cell corresponding to a range of process conditions. Based on the process conditions associated with each cell and a historical propensity toward corrosion under such process conditions, each cell is assigned a corrosion risk factor of low risk, medium risk, or high risk. If process conditions lead to identification of medium risk or high risk in either risk-based matrix, the process parameters may be adjusted until conditions leading to a low risk are reached.

Advantageously, the present disclosure allows corrosion-causing process conditions to be quickly identified and easily remedied. For example, it may be difficult in some cases to adjust the pH or oxygen content of a water stream, but it may be simpler to decrease the temperature or the conductivity to decrease the extent to which pH and oxygen content lead to corrosion. Moreover, the present disclosure allows the most pressing corrosion-causing conditions to be addressed quickly, even in near real-time, and possibly without simultaneously altering other corrosion-causing conditions.

Accordingly, methods of the present disclosure may comprise: monitoring a plurality of parameters in a water stream. The plurality of parameters comprise pH, temperature, oxygen content, and conductivity. The methods include assessing a first corrosion risk factor using a first risk-based matrix based upon pH as a function of temperature, followed by assigning the first corrosion risk factor as a pH low-risk, a pH medium-risk, or a pH high-risk. The methods also include assessing a second corrosion risk factor using a second risk-based matrix based upon oxygen content as a function of conductivity, followed by assigning the second corrosion risk factor as an oxygen content low-risk, an oxygen content medium-risk, or an oxygen content high-risk. Once the first and second corrosion risk factors have been determined, the methods include assessing an overall corrosion risk factor as a higher of the first corrosion risk factor and the second corrosion risk factor, the overall corrosion risk factor being an overall low-risk, an overall medium-risk, or an overall high-risk. In the case that the first corrosion risk factor and the second corrosion risk factor are the same (i.e., both a medium-risk or both a high-risk), the overall corrosion risk factor is designated as overall medium-risk or overall high-risk, respectively. Finally, if the overall risk factor is assessed as an overall high-risk or an overall medium-risk, at least one corrective action may be performed upon the water stream to decrease the overall risk factor.

In various embodiments, the water stream may be a sour water stream comprising at least hydrogen sulfide (H₂S). The location from which a sour water stream is obtained is not believed to be particularly limited. In non-limiting examples, a sour water stream may be obtained as an oilfield waste stream or as spent stripping water from an acid gas removal process.

The plurality of parameters being monitored to assess the risk of corrosion may represent data that is already being collected for the water stream as part of routine chemical process monitoring. For example, the temperature of the water stream may be obtained from a temperature indicator, such as a thermometer or thermocouple; the oxygen content of the water stream may be determined by an oxygen gas analyzer or similar oxygen-responsive sensor; the pH of the water stream may be determined using equipment such as a pH electrode; and conductivity may be measured using conventional conductivity probes. Advantageously, the methods of the present disclosure may leverage this data to provide an assessment of the risk of corrosion taking place.

Conductivity represents a material's ability to conduct electric current. Electric current is defined as a flow of charged particles, such as electrons or ions. Conductivity, also known as electrical conductivity or specific conductance, may be measured using conventional conductivity probes. Conductivity (electrical conductivity) is the reciprocal of electrical resistivity.

Monitoring of the plurality of parameters may be performed continuously or discontinuously. When performed discontinuously, monitoring of the process parameters may be performed at intervals determined by an operator. Common intervals for monitoring of the parameters may include, for example, 10 second intervals, 30 second intervals, 1 minute intervals, 2 minute intervals, 3 minute intervals, 5 minute intervals, 10 minute intervals, 20 minute intervals, 30 minute intervals, 1 hour intervals, or even longer intervals.

Low pH values may increase the risk of corrosion. The risk of corrosion may be decreased by raising the pH value. In addition, pH values are also affected by the temperature. As the temperature falls, pH rises. By lowering the temperature, the pH may rise above a corrosion-causing value. In addition, at lower temperatures, the reaction rate of hydrogen ions with a metal may decrease.

An excessively high oxygen content may increase the risk of corrosion. The risk of corrosion may be decreased by lowering the oxygen content. In addition, corrosion resulting from a high oxygen content is more pronounced at high conductivity values than at low conductivity values. By lowering the conductivity, the ability of a fixed amount of oxygen to promote corrosion may decrease.

In some cases, lowering the temperature of the water stream and/or lowering the conductivity of the water stream may be more straightforward than raising the pH or lowering the oxygen content of the water stream. Therefore, multiple parameters may be adjusted to attain conditions less likely to promote corrosion.

In various embodiments of the present disclosure, the first corrosion risk factor may be defined based upon the following temperature and pH conditions. The pH high-risk from the first corrosion risk factor is defined:
a. as a pH of about 4.5 or less for the water stream when the temperature of the water stream is about 100° C. or above, or
b. as a pH of about 4.5 to about 4.75 for the water stream when the temperature of the water stream is at about 125° C. or above.
   The pH medium-risk from the first corrosion risk factor is defined:
c. as a pH of about 4.5 or less for the water stream when the temperature of the water stream is about 100° C. or less,
d. as a pH of about 4.5 to about 4.75 for the water stream when the temperature of the water stream is about 100° C. to about 125° C., or
e. as a pH of about 4.75 to about 5.25 for the water stream when the temperature of the water stream is about 125° C. or above.
   The pH low-risk from the first corrosion risk factor is defined:
f. as a pH of about 4.5 to about 4.75 for the water stream when the temperature of the water stream is about 100° C. or below,
g. as a pH of about 4.75 to about 5.25 for the water stream when the temperature of the water stream is about 125° C. or below, or
h. as a pH of about 5.25 or above for the water stream.

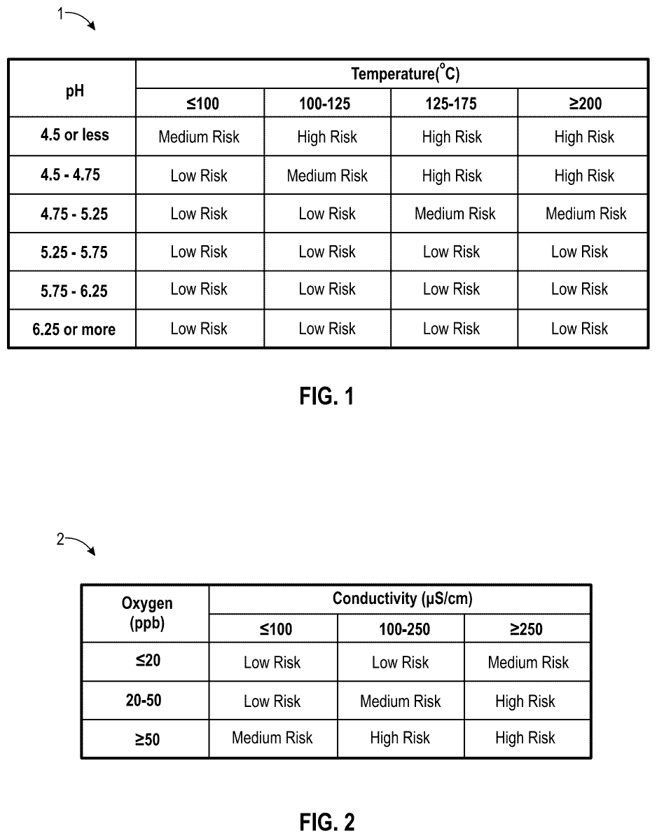

FIG. 1 is an illustrative example of a risk-based matrix based 1 on pH as a function of temperature. Risk-based matrix 1 is based upon the pH and temperature conditions specified immediately above. The assignment of a particular set of temperature and pH conditions as being pH high-risk, pH medium-risk, or pH low-risk may be based upon experimentally verified corrosion under such conditions.

In various embodiments of the present disclosure, the second corrosion risk factor may be defined based upon the following oxygen content and conductivity conditions. The oxygen content high-risk from the second corrosion risk factor is defined:
a. as an oxygen content of about 20 parts per billion (ppb) or more for the water stream when the conductivity of the water stream is about 250 microsiemens per centimeter (μS/cm) or more, or
b. as an oxygen content of about 50 ppb or more for the water stream when the conductivity of the water stream is about 100 μS/cm to about 250 μS/cm.
   The oxygen content medium-risk from the second corrosion risk factor is defined:
c. as an oxygen content of about 50 ppb or more for the water stream when the conductivity of the water stream is about 100 μS/cm or less,
d. as an oxygen content of about 20 ppb to about 50 ppb for the water stream when the conductivity of the water stream is about 100 μS/cm to about 250 μS/cm, or
e. as an oxygen content of about 20 ppb or less for the water stream when the conductivity of the water stream is about 250 μS/cm or more.
   The oxygen content low-risk from the second corrosion risk factor is defined:
f. as an oxygen content of about 50 ppb or less for the water stream when the conductivity of the water stream is about 100 μS/cm or less, or
g. as an oxygen content of about 20 ppb or less for the water stream when the conductivity of the water stream is about 100 μS/cm to about 250 μS/cm.

FIG. 2 is an illustrative example of a risk-based matrix 2 based on oxygen content as a function of conductivity. Risk-based matrix 2 is based upon the oxygen content and conductivity conditions specified immediately above. The assignment of a particular set of oxygen content and conductivity conditions as being oxygen content high-risk, oxygen content medium-risk, or oxygen content low-risk may be based upon experimentally verified corrosion under such conditions.

The overall corrosion risk factor may be determined as a higher of the first corrosion risk factor determined from risk-based matrix 1 and the second corrosion risk factor determined from risk-based matrix 2. Generally, the most serious risk factor is addressed first when altering the plurality of parameters to decrease the risk of corrosion. The two risk factors may contribute equally to corrosion (e.g., producing corrosion at about the same rate) or one risk factor may promote faster corrosion than does the other. The latter may be true even if both of the two risk factors are the same (i.e., both medium risk or both high risk). If the two risk factors of pH and oxygen content result in a tie (i.e., both medium risk or both high risk), then a corrective action is performed to lower the pH first, due to pH's significant effect on increasing the rate of corrosion.

If the overall risk factor is an overall high-risk or an overall medium-risk, at least one corrective action may be performed upon the water stream to decrease the overall risk factor. Depending on the risk factor that needs to be lowered, performing at least one corrective action may comprise raising the pH of the water stream, lowering the temperature of the water stream, decreasing the oxygen content of the water stream (e.g., by introducing an oxygen scavenger to the water stream), decreasing the conductivity of the water stream (e.g., by diluting the water stream), or any combination thereof. Thus, to mitigate corrosion risk from pH, the pH may be raised and/or the temperature may be lowered. In the case of lowering the temperature, a higher pH range may be tolerated without producing excessive corrosion. Similarly, to mitigate corrosion risk from oxygen content, the oxygen content may be lowered and/or the conductivity may be lowered. In the case of lowering the conductivity, a higher oxygen content may be tolerated without producing excessive corrosion.

If the overall risk factor is an overall high-risk or an overall medium-risk resulting from oxygen content (i.e., the second corrosion risk factor), the oxygen content may be lowered and/or the conductivity may be lowered.

Certain pH or conductivity conditions may be highly likely to lead to corrosion, regardless of the corresponding temperature or the corresponding oxygen content. If such conditions are identified, immediate corrective actions may be taken. For example, if the pH is about 4.5 or less (thereby leading to at least a pH medium-risk), the pH may be increased to within a range of at least 4.5 to 4.75, and possibly even higher depending on the corresponding temperature. Similarly, if the conductivity is 250 μS/cm or more (thereby leading to at least an oxygen content medium-risk), the conductivity may be decreased to within a range of at least about 100 μS/cm to 250 μS/cm, and possibly even lower depending on the corresponding oxygen content. Likewise, if the oxygen content is 50 ppb or more (thereby leading to at least an oxygen content medium-risk), the oxygen content may be decreased to within a range of about least 20 ppb to 50 ppb, and possibly even lower depending on the corresponding conductivity value.

Performing at least one corrective action may comprise introducing an oxygen scavenger into the water stream. For example, an oxygen scavenger may be introduced when the oxygen content of the water stream is 50 parts per billion (ppb) or more. When the oxygen content is 50 ppb or more, the second corrosion risk factor is at least a medium risk. By lowering the oxygen content to 50 ppb or below, it is at least possible to reach conditions having a low risk if the conductivity is also 100 μS/cm or less. If the conductivity is not 100 μS/cm or less, the oxygen content may be lowered further (e.g., by adding additional oxygen scavenger) and/or by decreasing the conductivity of the water stream (e.g., through dilution).

Oxygen scavengers, which may be an oxygen absorber, suitable for use in the present disclosure are not considered to be particularly limited. An oxygen scavenger is a substance that is used to reduce or completely remove oxygen in fluids and enclosed spaces to prevent oxygen-induced corrosion. Oxygen scavengers may reduce the amount of oxygen physically (e.g., by adsorption), chemically, or enzymatically. Examples of oxygen scavengers include, but are not limited to, metal oxides, ascorbic acid, ascorbic acid derivatives, catechol, catechol derivatives, sulfites, bisulfites, metabisulfites, hydroxylamines, thiols, hydroquinone, hydroquinone derivatives, carbazides, oximes, unsaturated hydrocarbon polymers, nylons (polyamides), hydrazines, enzymes, and any combination thereof.

Examples of metal oxides suitable for oxygen scavenging include, but are not limited to, iron oxides such as iron(II) oxide (ferrous oxide) (FeO), iron(III) oxide (ferric oxide) (Fe₂O₃), iron(II,III) oxide (Fe₃O₄), and any combination thereof.

Examples of ascorbic acid derivatives suitable for oxygen scavenging include, but are not limited to, ascorbates, isoascorbates, and any combination thereof. Isoascorbates are also known as erythorbates, one common example of which is sodium erythorbate.

Examples of sulfites suitable for oxygen scavenging include, but are not limited to, sodium sulfite (Na₂SO₃), potassium sulfite (K₂SO₃), lithium sulfite (Li₂SO₃), magnesium sulfite, and calcium sulfite (CaSO₃), barium sulfite (BaSO₃), and any combination thereof. Examples of suitable bisulfites include, but are not limited to, sodium bisulfite (NaHSO₃), potassium bisulfite (KHSO₃), lithium bisulfite (LiHSO₃), and any combination thereof. Examples of suitable metabisulfites include, but are not limited to, sodium metabisulfite (Na₂S₂O₅), potassium metabisulfite (K₂S₂O₅), and any combination thereof.

One nonlimiting example of a common hydroxylamine suitable for oxygen scavenging is diethylhydroxylamine.

Examples of thiols suitable for oxygen scavenging include, but are not limited to, glutathione, glutathione derivatives, cysteine, cysteine derivatives, and any combination thereof.

One nonlimiting example of a common carbazide for oxygen scavenging is carbohydrazide.

Suitable oximes for oxygen scavenging include both aldoximes and ketoximes. 2-Butanone oxime is an example of one suitable oxime for oxygen scavenging.

Examples of suitable unsaturated hydrocarbon polymers include, but are not limited to, polymyrcene, polyisoprene, polybutadiene, and combinations thereof.

Examples of hydrazines suitable for oxygen scavenging include, but are not limited to, hydrazine, 1,1-dimethylhydrazine, and 1,2-dimethylhydrazine.

Performing at least one corrective action may comprise diluting the water stream. Diluting the water stream may decrease the conductivity, which may thereby lower a risk factor associated with a high oxygen content. For example, dilution may take place when the conductivity of the water stream is 250 μS/cm or more. When the conductivity is 250 μS/cm or more, the second corrosion risk factor is at least a medium risk. By lowering the conductivity to 250 μS/cm or less, it is at least possible to reach conditions having a low risk if the oxygen content is also 20 ppb or less. If the conductivity needs to be lowered further, additional dilution may take place and/or the oxygen content may be lowered further by introducing an oxygen scavenger.

Dilution of the water stream may be conducted using any water source that contains a sufficiently low concentration of ions to decrease the conductivity when combined with the water stream. Additional criteria for suitable water sources include those that do not contribute excessive acidity, which might otherwise increase the first corrosion risk factor while still effectively mitigating the second corrosion risk factor. For example, the conductivity of the water stream may be lowered by the addition of chilled water (e.g., 4° C. to 20° C.), wherein the chilled water may also decrease the temperature of the water stream and potentially decrease the first corrosion risk factor as well. In some embodiments, the conductivity of the water stream may be lowered by adding stripped sour water to the water stream. Stripped sour water refers to a portion of a sour water stream that has had at least a portion of its hydrogen sulfide removed through a suitable technique.

If the first corrosion risk factor is either a pH medium-risk or a pH high-risk, performing at least one corrective action may comprise raising the pH of the water stream by adding a neutralization reagent, lowering the temperature of the water stream, or any combination thereof.

If the overall corrosion risk factor is an overall high-risk or an overall medium-risk resulting from pH (i.e., the first corrosion risk factor), the pH may be increased and/or the temperature may be lowered. Lowering the temperature of the water stream may include, but is not limited to, activation of a fan, passing the water through a heat exchanger, and/or adding cold water using a water pump. Raising the pH of the water stream may be conducted by adding a neutralization reagent. The neutralization reagent may be added in an isolated form (e.g., as a solid or a liquid), or the neutralization reagent may be introduced in an aqueous phase.

Suitable neuralization reagents include any compound that increases the pH of the water stream by neutralizing acid. Examples of suitable neutralization reagents include, but are not limited to, alkali metal carbonates, alkali metal bicarbonates, alkaline earth metal carbonates, alkaline earth metal bicarbonates, alkali metal hydroxides, alkaline earth metal hydroxides, and any combination thereof.

Alkali metal carbonates comprise an alkali metal and a carbonate (CO₃²⁻) group Examples of suitable alkali metal carbonates include, but are not limited to, lithium carbonate (Li₂CO₃), sodium carbonate (Na₂CO₃), potassium carbonate (K₂CO₃), rubidium carbonate (Rb₂CO₃), cesium carbonate (Cs₂CO₃), and any combination thereof.

Alkali metal bicarbonates comprise an alkali metal and a bicarbonate (hydrogen carbonate) group (HCO₃⁻). Examples of suitable alkali metal bicarbonates include, but are not limited to, lithium bicarbonate (LiHCO₃), sodium bicarbonate (NaHCO₃), potassium bicarbonate (KHCO₃), rubidium bicarbonate (RbHCO₃), cesium bicarbonate (CsHCO₃), and any combination thereof.

Alkaline earth metal carbonates comprise an alkaline earth metal and a carbonate (CO²⁻₃) group. Examples of suitable alkaline earth metal carbonates include, but are not limited to, beryllium carbonate (BeCO₃), magnesium carbonate (MgCO₃), calcium carbonate (CaCO₃), strontium carbonate (SrCO₃), barium carbonate (BaCO₃), and any combination thereof.

Alkaline earth metal bicarbonates comprise an alkaline earth metal and two bicarbonate (hydrogen carbonate) groups. Examples of suitable alkaline earth metal bicarbonates, include, but are not limited to, magnesium bicarbonate, calcium bicarbonate, and any combination thereof.

Alkali metal hydroxides comprise an alkali metal and a hydroxide (⁻OH) group. Examples of alkali metal hydroxides include, but are not limited to, lithium hydroxide (LiOH), sodium hydroxide (NaOH), potassium hydroxide (KOH), rubidium hydroxide (RbOH), cesium hydroxide (CsOH), and any combination thereof.

Alkaline earth metal hydroxides comprise an alkaline earth metal and two hydroxide (⁻OH) groups. Examples of suitable alkaline earth metal hydroxides include, but are not limited to, beryllium hydroxide (Be(OH)₂), magnesium hydroxide (Mg(OH)₂), calcium hydroxide (slaked lime) (Ca(OH)₂), strontium hydroxide (Sr(OH)₂), barium hydroxide (Ba(OH)₂), and any combination thereof.

If the first corrosion risk factor is either a pH medium-risk or a pH high-risk, performing at least one corrective action may comprise raising the pH of the water stream by adding a neutralization reagent, lowering the temperature of the water stream, or any combination thereof. Such corrective actions may be performed when a first cell of the first risk-based matrix originally characterizes the water stream as having a pH medium-risk or a pH high-risk. A second cell having a pH low-risk nearest the first cell is reached upon performing the at least one corrective action. When performing at least one corrective action in this manner, the least disruptive set of changes needed to move the pH and temperature conditions into a pH low-risk cell of the first risk-based matrix are performed. Movement from the first cell to the second cell in the first risk-based matrix may occur horizontally, vertically, diagonally, or any combination thereof.

If the second corrosion risk factor is an oxygen content medium-risk or an oxygen content high-risk, performing at least one corrective action may comprise introducing an oxygen scavenger to the water stream, diluting the water stream, or any combination thereof. Such corrective actions may be performed when a first cell of the second risk-based matrix originally characterizes the water stream as having an oxygen content medium-risk or an oxygen content high-risk. A second cell having an oxygen content low-risk nearest the first cell is reached upon performing the at least one corrective action. When performing at least one corrective action in this manner, the least disruptive set of changes needed to move the oxygen content and conductivity conditions into an oxygen content low-risk cell of the second risk-based matrix is performed. Movement from the first cell to the second cell in the second risk-based matrix may occur horizontally, vertically, diagonally, or any combination thereof.

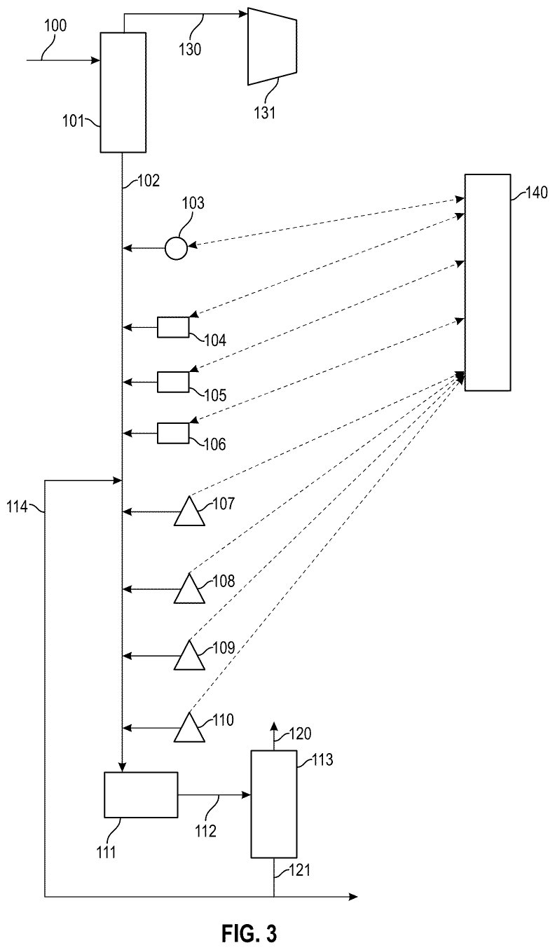

FIG. 3 is a diagram of an illustrative system for processing a water stream according to embodiments of the present disclosure. Control system 140 utilizes multiple measured parameters to determine what, if any, corrective actions need to be taken to decrease the risk of corrosion. Control system 140 is capable of either automatic or manual operations. In the depicted system and accompanying description, it is assumed that a sour water stream is being processed. Other water streams prone to producing corrosion may be processed in a related manner.

Referring to FIG. 3, wet acid gas flows through gas pipe 100 to knockout drum 101. Knockout drum 101 removes sour water as a bottoms stream via water pipe 102. Free acid gas flows from knockout drum 101 via gas pipe 130 to compressor 131.

Pumps 104-106 may each supply a reagent to the sour water stream in water pipe 102. Pumps 104-106 are communicatively coupled to controller 140. Controller 140 may determine which reagent(s) need(s) to be added based upon physical parameters measured within the sour water stream and utilizing the risk-based matrices described herein. Controller 140 may similarly activate cooling fan 103, which may be on or off depending on the temperature conditions needed in the sour water stream. In non-limiting examples, pump 104 may introduce a neutralizer, pump 105 may introduce an oxygen scavenger, and pump 106 may introduce cold water to the sour water stream in water pipe 102.

Sensors 107-110 may monitor parameters in the sour water stream. Multiple arrangements of sensors 107-110 are possible, one nonlimiting embodiment being described here. Sensor 107 may be a pH sensor, sensor 108 may be a temperature sensor, sensor 109 may be an oxygen gas sensor, and sensor 110 may be a conductivity sensor. Data provided by sensors 107-110 is communicated to control system 140, which may utilize the data for decreasing corrosion risk, as described above.

The sour water stream then flows to separator 111. Separator 111 removes additional acid gas as an overhead stream, and the water then flows through water pipe 112 to sour water stripper 113. Additional hydrogen sulfide is removed in sour water stripper 113 via gas line 120, and stripped sour water exits via gas line 120. If desired, a portion of the stripped sour water can be recycled via water pipe 114 to provide dilution of the sour water stream in water pipe 102. Although not shown, water pipe 114 may recycle the stripped sour water to pump 106 instead of introducing the stripped sour water at a separate location.

Collectively, sensor 107 and sensor 108 acquire data (pH and temperature) for determining the first corrosion risk factor. Collectively, sensor 109 and sensor 110 acquire data (oxygen content and conductivity) for determining the second corrosion risk factor. The data is communicated to control system 140, either in a wired or wireless manner, which then determines the first corrosion risk factor, the second corrosion risk factor, and the overall corrosion risk factor. Control system 140 then actuates any of cooling fan 103, or pumps 104-106 to perform corrective actions as needed.

The present disclosure is further directed to the following non-limiting clauses:
Clause 1. A method comprising: monitoring a plurality of parameters in a water stream; wherein the plurality of parameters comprises pH, temperature, oxygen content, and conductivity; assessing a first corrosion risk factor using a first risk-based matrix based upon pH as a function of temperature; assigning the first corrosion risk factor as a pH low-risk, a pH medium-risk, or a pH high-risk; assessing a second corrosion risk factor using a second risk-based matrix based upon oxygen content as a function of conductivity; assigning the second corrosion risk factor as an oxygen content low-risk, an oxygen content medium-risk, or an oxygen content high-risk; assessing an overall corrosion risk factor as a higher of the first corrosion risk factor and the second corrosion risk factor, the overall corrosion risk factor being an overall low-risk, an overall medium-risk, or an overall high-risk; wherein if the first corrosion risk factor and the second corrosion risk factor are both a medium-risk, the overall corrosion risk factor is designated as overall medium-risk, and if the first corrosion risk factor and the second corrosion risk factor are both a high-risk, the overall corrosion risk factor is designated as overall high-risk; and if the overall risk factor is assessed as an overall high-risk or an overall medium-risk, performing at least one corrective action upon the water stream to decrease the overall risk factor.
Clause 2. The method of clause 1, wherein the water stream is a sour water stream comprising at least hydrogen sulfide (H₂S).
Clause 3. The method of clause 1 or clause 2, wherein monitoring the plurality of parameters takes place continuously.
Clause 4. The method of any one of clauses 1-3, wherein performing at least one corrective action comprises raising the pH of the water stream, lowering the temperature of the water stream, decreasing the oxygen content of the water stream, decreasing the conductivity of the water stream, or any combination thereof.
Clause 5. The method of any one of clauses 1-4, wherein performing at least one corrective action comprises introducing an oxygen scavenger into the water stream when the oxygen content is about 50 parts per billion or above.
Clause 6. The method of any one of clauses 1-5, wherein performing at least one corrective action comprises diluting the water stream when the conductivity is about 250 microsiemens per centimeter or above.
Clause 7. The method of clause 6, wherein the water stream is diluted with stripped sour water.
Clause 8. The method of any one of clauses 1-7, wherein performing at least one corrective action comprises raising the pH of the water stream when the pH is about 4.5 or below.
Clause 9. The method of any one of clauses 1-8, wherein if the overall risk factor is a pH medium-risk or a pH high-risk, performing at least one corrective action comprises raising the pH of the water stream by adding a neutralization reagent, lowering the temperature of the water stream, or any combination thereof.
Clause 10. The method of clause 9, wherein a first cell of the first risk-based matrix originally characterizes the water stream, and a second cell having a pH low-risk risk nearest the first cell is reached upon performing the at least one corrective action.
Clause 11. The method of any one of clauses 1-10, wherein if the overall risk factor is an oxygen content medium-risk or oxygen content high-risk, performing at least one corrective action comprises introducing an oxygen scavenger to the water stream, diluting the water stream, or any combination thereof.
Clause 12. The method of clause 11, wherein a first cell of the second risk-based matrix originally characterizes the water stream, and a second cell having an oxygen content low-risk risk nearest the first cell is reached upon performing the at least one corrective action.
Clause 13. The method of any one of clauses 1-12, wherein the pH high-risk from the first corrosion risk factor is defined as a pH of about 4.5 or less when the temperature is about 100° C. or above, or as a pH of about 4.5 to about 4.75 when the temperature is about 125° C. or above; wherein the pH medium-risk from the first corrosion risk factor is defined as a pH of about 4.5 or less when the temperature is about 100° C. or less, as a pH of about 4.5 to about 4.75 when the temperature is about 100° C. to about 125° C., or as a pH of about 4.75 to about 5.25 when the temperature is about 125° C. or above; and wherein the pH low-risk from the first corrosion risk factor is defined as a pH of about 4.5 to about 4.75 when the temperature is about 100° C. or below, as a pH of about 4.75 to about 5.25 when the temperature is about 125° C. or less, or as a pH of about 5.25 or above.
Clause 14. The method of any one of clauses 1-13, wherein the oxygen content high-risk from the second corrosion risk factor is defined as an oxygen content of about 20 parts per billion (ppb) or more when the conductivity is about 250 microsiemens per centimeter (μS/cm) or more, or as an oxygen content of about 50 ppb or more when the conductivity is about 100 μS/cm to about 250 μS/cm; wherein the oxygen content medium-risk from the second corrosion risk factor is defined as an oxygen content of about 50 ppb or more when the conductivity is about 100 μS/cm or less, as an oxygen content of about 20 ppb to about 50 ppb when the conductivity is about 100 μS/cm to about 250 μS/cm, or as an oxygen content of about 20 ppb or less when the conductivity is about 250 μS/cm or more; and wherein the oxygen content low-risk from the second corrosion risk factor is defined as an oxygen content of about 50 ppb or less when the conductivity is about 100 μS/cm or less, or as an oxygen content of about 20 ppb or less when the conductivity is about 100 μS/cm to about 250 μS/cm.
Clause 15. The method of any one of clauses 1-14, further comprising: after performing the at least one corrective action upon the water stream to decrease the overall risk factor, determining whether either the first corrosion risk factor or the second corrosion risk factor not selected as the overall risk factor remains or has become a medium-risk or high-risk; and performing at least one additional corrective action upon the water stream.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains,” “containing,” “includes,” “including,” “comprises,” and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

While the present disclosure has been described with respect to a limited number of embodiments, those skilled in the art, having benefit of this disclosure, will appreciate that other embodiments may be devised which do not depart from the scope of the disclosure as described herein. Accordingly, the scope of the disclosure should be limited only by the attached claims.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by one or more embodiments described herein. At the very least, and not as an attempt to limit the application of the doctrine of equivalents to the scope of the claim, each numerical parameter should at least be construed in light of the number of reported significant digits and by applying ordinary rounding techniques.

The invention claimed is:

1. A method comprising:
monitoring a plurality of parameters in a water stream;
wherein the plurality of parameters comprises pH, temperature, oxygen content, and conductivity;
assessing a first corrosion risk factor using a first risk-based matrix based upon pH as a function of temperature;
assigning the first corrosion risk factor as a pH low-risk, a pH medium-risk, or a pH high-risk;
assessing a second corrosion risk factor using a second risk-based matrix based upon oxygen content as a function of conductivity;
assigning the second corrosion risk factor as an oxygen content low-risk, an oxygen content medium-risk, or an oxygen content high-risk;
assessing an overall corrosion risk factor as a higher of the first corrosion risk factor and the second corrosion risk factor, the overall corrosion risk factor being an overall low-risk, an overall medium-risk, or an overall high-risk;
wherein if the first corrosion risk factor and the second corrosion risk factor are both a medium-risk, the overall corrosion risk factor is designated as overall medium-risk, and if the first corrosion risk factor and the second corrosion risk factor are both a high-risk, the overall corrosion risk factor is designated as overall high-risk; and
if the overall risk factor is assessed as an overall high-risk or an overall medium-risk, performing at least one corrective action upon the water stream to decrease the overall risk factor.

2. The method of claim 1, wherein the water stream is a sour water stream comprising at least hydrogen sulfide (H₂S).

3. The method of claim 2, wherein monitoring the plurality of parameters takes place continuously.

4. The method of claim 2, wherein performing at least one corrective action comprises raising the pH of the water stream, lowering the temperature of the water stream, decreasing the oxygen content of the water stream, decreasing the conductivity of the water stream, or any combination thereof.

5. The method of claim 2, wherein performing at least one corrective action comprises introducing an oxygen scavenger into the water stream when the oxygen content is about 50 parts per billion or above.

6. The method of claim 2, wherein performing at least one corrective action comprises diluting the water stream when the conductivity is about 250 microsiemens per centimeter or above.

7. The method of claim 6, wherein the water stream is diluted with stripped sour water.

8. The method of claim 2, wherein performing at least one corrective action comprises raising the pH of the water stream when the pH is about 4.5 or below.

9. The method of claim 2, wherein if the overall risk factor is a pH medium-risk or a pH high-risk, performing at least one corrective action comprises raising the pH of the water stream by adding a neutralization reagent, lowering the temperature of the water stream, or any combination thereof.

10. The method of claim 9, wherein a first cell of the first risk-based matrix originally characterizes the water stream, and a second cell having a pH low-risk risk nearest the first cell is reached upon performing the at least one corrective action.

11. The method of claim 2, wherein if the overall risk factor is an oxygen content medium-risk or oxygen content high-risk, performing at least one corrective action comprises introducing an oxygen scavenger to the water stream, diluting the water stream, or any combination thereof.

12. The method of claim 11, wherein a first cell of the second risk-based matrix originally characterizes the water stream, and a second cell having an oxygen content low-risk risk nearest the first cell is reached upon performing the at least one corrective action.

13. The method of claim 2, wherein the pH high-risk from the first corrosion risk factor is defined as a pH of about 4.5 or less when the temperature is about 100° C. or above, or as a pH of about 4.5 to about 4.75 when the temperature is about 125° C. or above;
wherein the pH medium-risk from the first corrosion risk factor is defined as a pH of about 4.5 or less when the temperature is about 100° C. or less, as a pH of about 4.5 to about 4.75 when the temperature is about 100° C. to about 125° C., or as a pH of about 4.75 to about 5.25 when the temperature is about 125° C. or above; and
wherein the pH low-risk from the first corrosion risk factor is defined as a pH of about 4.5 to about 4.75 when the temperature is about 100° C. or below, as a pH of about 4.75 to about 5.25 when the temperature is about 125° C. or less, or as a pH of about 5.25 or above.

14. The method of claim 2, wherein the oxygen content high-risk from the second corrosion risk factor is defined as an oxygen content of about 20 parts per billion (ppb) or more when the conductivity is about 250 microsiemens per centimeter (μS/cm) or more, or as an oxygen content of about 50 ppb or more when the conductivity is about 100 μS/cm to about 250 μS/cm;
wherein the oxygen content medium-risk from the second corrosion risk factor is defined as an oxygen content of about 50 ppb or more when the conductivity is about 100 μS/cm or less, as an oxygen content of about 20 ppb to about 50 ppb when the conductivity is about 100 μS/cm to about 250 μS/cm, or as an oxygen content of about 20 ppb or less when the conductivity is about 250 μS/cm or more; and
wherein the oxygen content low-risk from the second corrosion risk factor is defined as an oxygen content of about 50 ppb or less when the conductivity is about 100 μS/cm or less, or as an oxygen content of about 20 ppb or less when the conductivity is about 100 μS/cm to about 250 μS/cm.

15. The method of claim 2, further comprising:
after performing the at least one corrective action upon the water stream to decrease the overall risk factor, determining whether either the first corrosion risk factor or the second corrosion risk factor not selected as the overall risk factor remains or has become a medium-risk or high-risk; and
performing at least one additional corrective action upon the water stream.
